(19) United States
(12) Patent Application Publication
(10) Pub. No.: US 2013/0014316 A1
(43) Pub. Date: Jan. 17, 2013

(54) DETACHABLE FACEMASK FRAME AND FACEMASK FOR A GOGGLE

(71) Applicants: James Nelson Castro, Laguna Niguel, CA (US); Hans Karsten Moritz, Foothill Ranch, CA (US); Casey Aaron Jones, Oceanside, CA (US)

(72) Inventors: James Nelson Castro, Laguna Niguel, CA (US); Hans Karsten Moritz, Foothill Ranch, CA (US); Casey Aaron Jones, Oceanside, CA (US)

(73) Assignee: Oakley, Inc., Foothill Ranch, CA (US)

(21) Appl. No.: 13/184,453

(22) Filed: Jul. 15, 2011

(51) Int. Cl. A61F 9/02; A41D 13/11

(52) U.S. Cl. 2/424; 2/427

(57) ABSTRACT

Facial coverings and eyewear systems are provided herein for improving the ease and comfort of wearing facemasks with a goggle. In some embodiments, a facemask frame is provided that can removably attach to a facemask or face covering to a goggle. In some embodiments, a separate facemask, such as a cloth covering, can be provided. In some embodiments, a facemask can be interconnected with the facemask frame to secure the facemask relative to the goggle during use for covering a portion of the wearer's face.

### BACKGROUND

1. Field of the Inventions

The present disclosure relates generally to facial coverings and eyewear, and more particularly to a facemask frame and system that can removably attach to a facemask or face covering to a goggle, and to a facemask system that controls airflow through a facemask.

2. Description of the Related Art

A wide variety of improvements have been made in recent years in the eyewear field, particularly with respect to eyewear intended for use in active sports. Protective helmet shields or visors are known for such activities as motorcycle riding, football, lacrosse, hockey and the like. Goggles are used for various activities, including skiing, motocross, underwater diving masks, and a variety of industrial safety applications such as welding and for power equipment operators. Typically, goggles offer sealed protection to the eyes and adjacent areas of the wearer's face against particulate matter or moisture, but leave a lower portion of the face, chin, and neck exposed.

Supplemental face, chin, and neck protection such as scarves and handkerchiefs are used to cover the lower portion of the face, chin, and neck, but leave a gap between the supplemental protection and the goggles, exposing portions of the face and/or neck. In some instances, a handkerchief may be tucked under the padding of the goggle near the nose of the wearer, but this can reduce the seal and comfort of the goggle against the face.

### SUMMARY

There is provided in accordance with one aspect of the present inventions, a facemask frame system for attaching a facemask to a goggle. The facemask frame system comprises a frame nosepiece, and a first and second pair of opposed arms extending from the frame nosepiece. The frame nosepiece is configured to cover a portion of a goggle nosepiece. The first pair of opposed arms extends from the frame nosepiece. The first pair of opposed arms is configured to removably engage the frame nosepiece with an anterior-facing portion of a goggle frame proximate to the goggle nosepiece. The second pair of opposed arms extends laterally from the frame nosepiece. The second pair of opposed arms is configured to removably engage with a facemask. The second pair of opposed arms is configured to support a portion of an upper edge of the facemask and extend laterally along the goggle frame when the first pair of opposed arms are engaged with the goggle frame and the second pair of opposed arms are engaged with the facemask.

In some embodiments, the second pair of opposed arms can be configured to extend laterally along an anterior-facing portion of the goggle frame when the first pair of opposed arms are engaged with the goggle frame and the second pair of opposed arms are engaged with the facemask. The second pair of opposed arms can be configured to form a seal against the goggle frame with the portion of the facemask when the first pair of opposed arms are engaged with the goggle frame and the second pair of opposed arms are engaged with the facemask. The second pair of opposed arms can be configured to extend along and form a seal against the goggle frame without mechanically attaching to the goggle frame when the first pair of opposed arms are engaged with the goggle frame and the second pair of opposed arms are engaged with the facemask. The second pair of opposed arms can be configured to be biased against the goggle frame when the first pair of opposed arms are engaged with the goggle frame and the second pair of opposed arms are engaged with the facemask.

In some embodiments, the first and the second pair of opposed arms can be spaced apart in an anterior-posterior direction with respect to each other by a distance D. The distance D can be approximately within a range of between about at least 0.25 inches and/or less than or equal to about 1.25 inches. The facemask frame system can further comprise the goggle, wherein the distance D can be approximately the distance between the anterior-facing portion of the goggle frame proximate to the goggle nosepiece and an anterior-facing surface of a lip extending along a perimeter formed by the goggle frame. The lip can extend along a posterior perimeter formed by the goggle frame.

In some embodiments, each of the first pair of opposed arms can further comprise a posterior-extending portion with a goggle engagement element configured to removably engage with the anterior-facing portion of the goggle frame. The goggle engagement elements can be positioned at the approximate distal end of the posterior-extending portions. The facemask frame system can further comprise the goggle, wherein the goggle engagement elements can be configured to engage with corresponding facemask frame engagement elements positioned on the anterior-facing portion of the goggle frame. The facemask frame engagement elements can comprise a pair of recesses formed on opposed sides of the goggle nosepiece. The facemask frame system can further comprise a pair of inserts configured to be received by and engage with the pair of recesses, the inserts further configured to receive the goggle engagement elements, to provide the engagement of the goggle engagement elements with the recesses.

In some embodiments, the facemask frame system can further comprise the goggle and the facemask.

In some embodiments, each of the second pair of opposed arms can include a first portion extending in a posterior direction with respect to the frame nosepiece, and a second portion extending laterally from the first portion. The second pair of opposed arms are spaced apart from the frame nosepiece by a distance D corresponding approximately to a distance between an anterior-facing surface of the goggle nosepiece and an anterior-facing surface of a lip extending along the posterior edge of the goggle frame. The distance D can be approximately 0.5 inches.

In some embodiments, the facemask frame system can further comprise a slot extending through the frame nosepiece. The slot can be configured to receive a strap configured to removably engage the facemask with the frame nosepiece.

In some embodiments, a method is provided of attaching a facemask to a goggle. A goggle can be provided that includes a frame with a medial recess configured to cover a portion of a wearer's nose. A facemask frame can be provided that includes a frame nosepiece configured to cover a portion of the medial recess, a first pair of opposed arms extending from the frame nosepiece, and a second pair of opposed arms extending laterally from the frame nosepiece, the second pair of opposed arms attached to the frame nosepiece posteriorly with respect to the first pair of opposed arms. A facemask can be provided with one or more engagement elements extending along a portion of an upper edge of the facemask, the engagement elements configured to engage with the second pair of opposed arms. The facemask can be removably engaged with the facemask frame by engaging the second pair of opposed arms with the one or more engagement elements of the facemask. The first pair of opposed arms can be removably engaged with the goggle frame proximate to the goggle nosepiece, such that the second pair of opposed arms support the portion of the upper edge of the facemask along the goggle frame, with a portion of the facemask positioned between the opposed arms and the goggle frame.

In some embodiments, removably engaging the first pair of opposed arms with the goggle frame can comprise forming a seal between the second pair of arms and the goggle frame.

In some embodiments, a kit is provided for attaching a facemask to a goggle. The kit comprises a pair of inserts and a facemask frame. The inserts are configured to be received by and engage with a pair of recesses positioned on an anterior portion of a goggle frame. The facemask frame can comprise a frame nosepiece, a pair of attachment elements, and a second pair of opposed arms. The frame nosepiece is configured to cover a portion of a nosepiece on a goggle frame. The pair of attachment elements extends from the frame nosepiece, the attachment elements configured to removably engage with the inserts. The second pair of opposed arms extends laterally from the frame nosepiece and is configured to removably engage with a facemask.

In some embodiments, a removable facemask system for goggles is provided, comprising a goggle, a facemask, and a facemask frame for attaching the facemask to the goggle. The goggle comprises a frame with a medial recess configured to cover a portion of a wearer's nose and a facemask frame engagement element configured on an anterior portion of the goggle frame proximate to the medial recess. The facemask comprises a channel extending along a portion of the facemask. The facemask frame comprises a frame nosepiece configured to cover a portion of the medial recess. The facemask frame further comprises a goggle engagement element extending from the frame nosepiece. The goggle engagement element is configured to removably engage with the facemask frame engagement element. The facemask frame further comprises a pair of opposed arms attached to the nosepiece. The pair of opposed arms comprise a laterally extending portion configured to be inserted into the channel of the facemask, such that the facemask is removably engaged with the facemask frame. The pair of opposed arms are posteriorly disposed on the frame nosepiece with respect to the goggle engagement elements.

There is provided in accordance with another aspect of the present inventions, a facemask system for goggles, comprising a facemask. The facemask comprises a body configured to wrap at least partially around a wearer's face, chin and/or neck. The facemask further comprises a pair of medially-facing, laterally-spaced, opposed openings extending through an upper portion of the facemask body. The openings are configured to removably engage with a pair of opposed arms extending laterally from a medial portion of a facemask frame or goggle.

In some embodiments, the facemask system for goggles further comprises the facemask frame, including the pair of opposed arms, the pair of opposed arms comprising a first pair of opposed arms. The facemask frame further comprises a frame nosepiece configured to cover a portion of a goggle nosepiece; and a second pair of opposed arms extending from the frame nosepiece. The second pair of opposed arms is configured to removably engage the frame nosepiece with an anterior-facing portion of a goggle frame proximate to the goggle nosepiece. The first pair of opposed arms are configured to support a portion of an upper edge of the facemask and extend laterally along the goggle frame when the second pair of opposed arms are engaged with the goggle frame and the first pair of opposed arms are engaged with the facemask through the pair of openings.

In some embodiments, the pair of openings extend laterally to form a pair of opposed channels along an upper edge of the facemask body, the channels configured to removably engage the pair of opposed arms.

In some embodiments, the facemask system for goggles further comprises one or more ventilation channels extending through a portion of the body.

In some embodiments, the body comprises a first body section and a second body section attached to each other along a hem. The hem includes a first hem portion spaced from a second hem portion such that at least one of the ventilation channels is formed between the first and second body sections and the first and second hem portions.

In some embodiments, a portion of the first body section extends under a portion of the second body section such that the portion of the second body section forms a flap that covers a portion of the at least one ventilation channels.

In some embodiments, the one or more ventilation channels comprises a plurality of ventilation openings forming a mesh.

In some embodiments, the facemask system for goggles further comprises a flap configured to be movable between an open position and a closed position with respect to at least one ventilation opening The flap allows less flow through the ventilation opening in the closed position with respect to the amount of flow the flap allows through the ventilation opening in the open position.

In some embodiments, the facemask system for goggles further comprises one or more attachment elements configured to attach the flap to a portion of the body in at least one of the open and closed positions.

In some embodiments, at least one of the one or more attachment elements comprises a magnet.

In some embodiments, the flap comprises a movable edge oriented in an approximately vertical position when the flap is in a closed position and the facemask is in an as-worn position.

In some embodiments, the movable edge is oriented in an approximately horizontal position when the flap is in an open position and the facemask is in an as-worn position.

In some embodiments, the flap further comprising a tab extending from an edge of the flap and configured to be grasped by a user to move the flap from the closed position to the open position.

There is provided in accordance with another aspect of the present inventions, a facemask system for goggles, comprising a facemask. The facemask comprises a body, a ventilation opening, a flap, and one or more attachment elements. The body is configured to wrap at least partially around a wearer's face, chin and/or neck. The ventilation opening extends through a portion of the body. The flap is configured to be movable between an open position and a closed position with respect to the ventilation opening. The flap allows less flow through the ventilation opening in the closed position with respect to the amount of flow the flap allows through the ventilation opening in the open position. The one or more attachment elements are configured to removably attach an edge of the flap to a portion body when the flap is in at least one of the open and closed position.

In some embodiments, at least one of the attachment elements comprises a magnet.

In some embodiments, the flap comprises a movable edge positioned in an approximately horizontal position when the facemask is in an as-worn position.

In some embodiments, the movable edge is configured to move between an upper position when the flap is in a closed position and a lower position when the flap is in an open position.

In some embodiments, the flap further comprising a tab extending from an edge of the flap and configured to be grasped by a user to move the flap from the closed position to the open position.

Further features and advantages of embodiments of the present inventions will become apparent from the detailed description of preferred embodiments which follows, when considered together with the attached drawings and claims.

### BRIEF DESCRIPTION OF THE DRAWINGS

These and other aspects of embodiments disclosed herein will be readily apparent from the following description and from the appended drawings (not necessarily to scale), which are meant to illustrate and not to limit the inventions, and in which:

FIG. 1 is a front and right perspective exploded view of an embodiment of a facemask frame for attaching a facemask to a goggle.

FIG. 2 is a front view of an embodiment of the facemask frame shown in FIG. 1 engaged with a facemask in a flat position.

FIG. 3 is a side view of an embodiment of the facemask frame shown in FIG. 1 engaged with a goggle and a facemask, in an as-worn position.

FIG. 4 is a front view of an embodiment of the facemask frame shown in FIG. 1 engaged with a goggle and a facemask, in an as-worn position.

FIGS. 5A-5D are front and right perspective, front, side, and top views, respectively, of an embodiment of the facemask frame shown in FIG. 1.

FIG. 6 is a partial side cross-sectional view of an embodiment of a facemask frame engaged with a goggle and a facemask taken along lines 6-6 of FIG. 4.

FIGS. 7A and 7B are partial front views of an embodiment of a facemask with a movable flap shown in a closed and opened position, respectively.

FIG. 8 is a front view of another embodiment of a facemask with a movable flap.

FIG. 9 is a front view of another embodiment of a facemask with a movable flap.

FIG. 10 is a front view of another embodiment of a facemask with a movable flap.

FIGS. 11A and 11B are front views of another embodiment of a facemask with a movable flap shown in a closed and opened position, respectively.

### DETAILED DESCRIPTION

Although preferred embodiments will be discussed below in terms of goggles particularly adapted for snow skiing or other winter sport applications, it will be understood that the inventions can also be employed with other eyewear applications, including other types of goggles, shields, helmet shields, military shields, eyeglasses or other products which protect the eyes, while leaving portions of the face, chin and neck exposed. Furthermore, while the illustrated embodiments exhibit particular lens surface geometries, front elevational shapes and orientations advantageous to the skiing application, the skilled artisan will readily be able to adapt the facemask frame systems disclosed herein to lenses having different geometries and orientations in the as-worn position beyond those illustrated herein. For example, the facemask frame system can be incorporated into eyewear with a single unitary lens, or dual (right/left) lenses with or without passive or active ventilation, and other known eyewear configurations. Additionally, while the embodiments of a facemask or facemask system disclosed herein are shown in combination with the facemask frame system and a goggle, the facemask or facemask system can be implemented without a goggle and/or facemask frame system.

Herein are described embodiments of an external and removable facemask frame. The facemask frame can be used to removably attach a variety of facial covering configurations (i.e., a scarf, covering, handkerchief or the like) for eyewear, and are not limited to the facemasks described herein. Such a configuration provides protection to a user's upper and lower face, eyes, chin, and neck, and reduces any exposed or unprotected areas formed between conventional goggles and supplemental face/neck protection. The facemask frame is removable from the goggle to allow a user to use the goggles without the facemask, for example, when the user is hot. The facemask is removable from the facemask frame to facilitate cleaning of the facemask, or to exchange various facemasks depending on weather conditions or preferences.

The facemask frame can include a nosepiece configured to removably engage with (e.g., seal) a corresponding nosepiece on a goggle. The frame can include one or more goggle engagement members that can be used to engage (e.g., snap fit, press fit, etc.) the facemask frame onto the goggle. The goggle engagement members can extend in a variety of directions from the frame nosepiece, including a lateral and/or posterior direction. The goggle engagement members can engage with corresponding facemask frame engagement elements or inserts on a goggle, positioned on opposing sides of the goggle nosepiece.

The facemask frame can also include one or more facemask engagement members configured to engage with and support a facemask attached to and extending from the facemask frame in a variety of directions. In some embodiments, a pair of facemask engagement members can extend in a lateral and/or posterior direction from the frame nosepiece. These members can be fitted into a channel, hem, loop, or other structure extending along a portion (e.g., a superior edge) of the facemask. Further, the frame nosepiece can comprise a slot configured to receive a strap extending from an upper portion of the facemask. The strap can be inserted into the slot, securing the strap and the facemask to the nosepiece.

In some embodiments, inserts (used for engagement of the goggle with the facemask frame) can be provided to retrofit an existing goggle for use with the facemask frame. In such an embodiment, the inserts are attached to the facemask frame engagement members on the goggle, and the facemask frame is attached to the inserts.

Also described herein are embodiments of a facemask system comprising a facemask that can be implemented separately from or in combination with the embodiments of the facemask frame and/or goggles described herein. In some embodiments, the facemask can include an attachment or engagement element configured to engage the facemask with a goggle. In some embodiments, the facemask can include an attachment element configured to engage the facemask with a facemask engagement member on a facemask frame which can attach to a goggle. In some embodiments, the engagement element can comprise a body with a pair of medially-facing opposed openings extending therethrough, the openings configured to removably engage with a portion of a facemask frame or goggle.

In some embodiments, the facemask system can include a flap configured to cover an opening, gap or channel extending through a portion of the facemask to provide ventilation or interior access to the wearer when the facemask system is in an as-worn position. The flap can be movable between an open and a closed position with respect to the opening, gap, or channel. The flap can be oriented to open and close in a vertical, horizontal, or angled direction when the facemask system is in an as-worn position. In such embodiments, the flap and/or an underlying layer of the facemask can include one or more attachment elements to allow a portion (e.g., an edge and/or corner) of the flap to be removably attached to the underlying layer, to secure the flap to the underlying layer in an open and/or closed position. In some such embodiments, the attachment element can comprise a magnet.

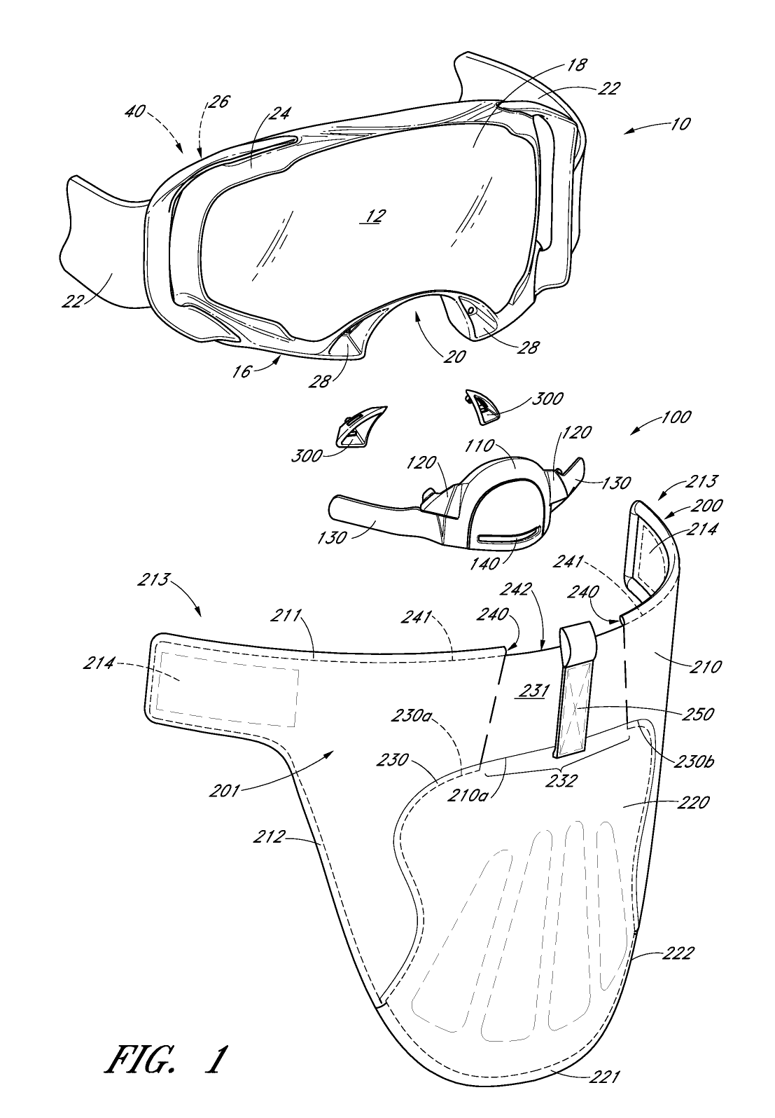

FIG. 1 illustrates a front and right perspective exploded view of an embodiment of a facemask frame 100 for attaching a facemask 200 to a goggle 10. Goggle 10 can comprise a single unitary lens construction, or can comprise one or more lenses, such as the illustrated double unitary lens construction. Goggle 10 can comprise a first, front or outer lens 12. In some configurations, the goggle 10 can further comprise a second, rear, posterior or inner lens, with an inter lens spacing between the outer lens 12 and the inner lens. Outer lens 12 can be separated from the inner lens in any of many different ways known in the art, such as a continuous gasket around the posterior perimeter of the outer lens 12 and the anterior perimeter of the inner lens. Lenses 12 is illustrated as a unitary lens, configured to extend across the vision of both eyes in an as-worn orientation upon a wearer's head, although other known configurations such as dual side by side lens systems, with or without separate orbitals, may be used with the facemask frame system.

The illustrated goggle 10 includes a mounting frame 16, configured to support the lens 12 on the wearer's head in the as-worn orientation. The illustrated frame 16 can extend continuously around the edges of the lenses, surrounding and defining a unitary viewing window 18 across which the lens 12 extends. The bottom edge of the frame 16 can include a medial recess or nosepiece 20 shaped to accommodate the wearer's nose when worn. While such a configuration is preferred for ski goggles, it is not essential for providing many of the advantages of the embodiments of facemasks and facemask frame systems described herein. In other arrangements, a frame may bound only the bottom edges of the lenses or only the top edges. Alternatively, a frame can bound any other portions of the lenses as will be evident to those of skill in the art. Frameless eyewear can also be constructed in accordance with certain aspects of embodiments disclosed herein. Alternatively, the frame may comprise a helmet, with one or more face shields, e.g., wherein lens 12 can comprise an outer and inner helmet shield to cover the front opening in said helmet. In the illustrated embodiment, though, the lens edges can be continuously bounded by the frame 16 of the goggle 10 as shown.

The frame 16 can comprise any of a variety of metals, composites or relatively rigid, molded thermoplastic materials which are well known in the art, and may be substantially transparent, translucent or opaque, or any of a variety of colors. The illustrated frame 16, however, can be preferably constructed of a relatively flexible yet elastic material, which can deform to facilitate application to the wearer's head and return to the original molded shape to retain the lens 12 in the desired orientation. Flexibility also facilitates a customized fit to the wearer's face. An exemplary frame material comprises urethane, though other polymeric materials are known which can satisfy these needs. The frame 16 can be manufactured by injection molding, machining and other construction techniques well known in the art.

A strap 22 can be attached to each lateral side of the frame 16, such as by attachment to outriggers in the form of lever arms that may be pivotably attached to the frame. Alternatively, the strap may be looped through slots provided at the lateral edges of the frame 16. In some configurations, the strap 22 can be adjustable and can comprise an elastic material to facilitate stretching over the wearer's head, and to facilitate pulling the frame 16 to conform against the wearer's face. It will be understood that, in other arrangements, the frame can be supported upon the wearer's head by multiple straps, by suspension from a helmet, or by a pair of earstems. Earstems can alternatively attach directly to the lenses or frame in a known fashion.

Frame 16 can have a double frame construction, including an outer or front frame portion 24, typically carrying the lens, and an inner or rear frame portion 26 spaced behind the front frame portion 24. The front portion and rear portion can be spaced from and connected to each other by a plurality of struts and/or ribs. The spacing can taper slightly from a maximum at the top of the goggle to a minimum at the bottom of the goggle 10. Bottom vents can be provided on the bottom surfaces of the front frame portion 24 and the rear frame portion 26 (e.g., inferior to and to the left and to the right of the medial recess 20) to provide passive ventilation to goggle 10 and prevent fogging. Each of the vents can be upwardly recessed (e.g., in a superior direction) relative to the bottom surfaces of frame portions 24, 26. Alternatively or additionally, side vents can be provided on the side surfaces of frame portions 24, 26, and can be inwardly (e.g., medially) recessed relative to the side surfaces. Alternatively or additionally, top vents can be provided on the top surfaces of frame portions 24, 26 and can be downwardly recessed (e.g., in an inferior direction) relative to the top surfaces.

A double frame structure thus can define relatively open lateral portions covered by lateral filter membranes and a relatively open top portion covered by a top membrane between the struts. Each of these membranes preferably can comprise a thin, air permeable material which can keep particles of snow or ice out, while allowing water vapor to pass through. An exemplary membrane material comprises an open cell polyurethane foam.

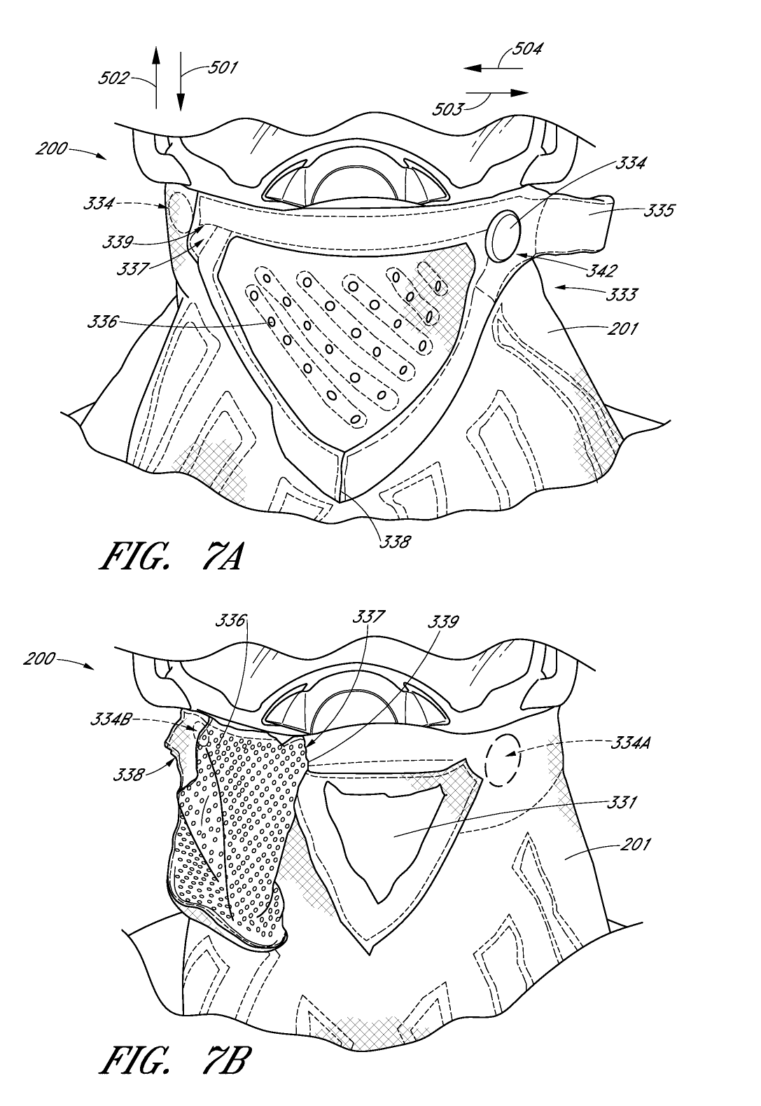

The rear surface of frame 16 (e.g., the rear frame portion 26) is preferably lined with a cushioning material 40 (FIGS. 4 and 6). The cushioning material 40 may comprise various layers, including, for example, an inner layer, an intermediate layer, and an outer layer. The inner layer may comprise a soft, matted or woven fiber to prevent chaffing the wearer's face. The intermediate layer preferably comprises a resilient, deformable material such as an open cell polyurethane foam, while the outer layer preferably can comprise a relatively more dense foam material which is readily adhered to the material of the rear frame portion 26. Further embodiments and details of a goggle with the aforementioned double frame construction, ventilation, cushioning material, and a variety of other structures that can be implemented with facemask frame 100 and/or facemask 200 are provided in co-pending U.S. patent application Ser. No. 12/960,083, the entirety of which is hereby expressly incorporated by reference herein.

In some embodiments, goggle 10 can comprise a lip, flange, shoulder, or similar structure, such as a lip 32 (FIG. 6) extending along the perimeter of frame 16. Lip 32 and frame 16 can be separately formed and attached to each other using a variety of the attachment methods described herein, or can be a solid or unitary component. Lip 32 can be extend along the perimeter of frame 16 at various positions along the width of frame 16. For example, lip 32 can extend along the anterior-most or posterior-most perimeter of frame 16, or any point therebetween. Lip 32 can provide additional support and surface area on the posterior surface of frame 16 for attaching cushion 40. Lip 32 can provide additional flexibility to the edges of frame 16, to provide improved sealing and/or conformance of the posterior side of frame 16 to a user's face. In some embodiments, lip 32 can provide an anterior-facing surface that can engage or seal with a portion of facemask 200 when facemask frame 100 is engaged with goggle 10 and facemask 200, as described further herein.

Frame 16 can include one or more facemask frame engagement or attachment elements configured to engage, and preferably, removably engage, goggle 10 with facemask frame 100. The engagement elements can comprise any of a variety of engagement and attachment structures and methods known or described herein, including adhesive, snaps, buttons, hooks, tabs, buttons, a press fit, buckles, zippers, clasps, interference fit, snap fit, slots, grooves, screws, rivets, magnets, hook and loop systems (e.g., VELCRO®), and the like. The facemask frame engagement elements can be positioned along various portions of goggle 10, and preferably, are positioned proximate to medial recess 20. The facemask frame engagement elements can comprise a pair of engagement elements positioned at an inferior or bottom portion of the frame 16, and/or laterally on either side of the medial recess 20. Preferably, the facemask frame engagement elements can be positioned on an anterior-facing portion of frame 16. In the illustrated embodiment, the facemask frame engagement elements comprise one or more recesses 28 extending into an anterior surface of frame 16. In some embodiments, recesses 28 can comprise an existing pair of front air intakes or vents, similar to the aforementioned vents in goggle 10. Recesses 28 can be any suitable shape to receive a corresponding goggle engagement element or member on facemask frame 100, and are not limited to the approximately triangular shape shown in FIG. 1. In some embodiments, recesses 28 comprise one or more edges that conform to the shape of lens 12, the bottom edge of frame 16, and/or the edge of recess 20.

The facial coverings described herein, some of which can be implemented with facemask frame 100 and/or goggle 10, can comprise any of a variety of rigid, semi-rigid, or preferably, flexible material capable of covering a portion of a wearer's face, chin, upper chest, and/or neck. It will be understood that in some embodiments, the facial coverings described herein can extend to also cover a portion of a wearer's head (such as a facemask that is integrated with a hat or hood).

The facial covering can comprise a durable, lightweight, breathable, water resistant, and/or washable material that can be used in many different environmental conditions, such as nylon, cotton, or the like. In some embodiments, the facial covering can comprise an insulating material to provide additional warmth to the wearer. In some embodiments, the facial covering can comprise a material that is elastic and/or conforms to, compresses, and/or fits snugly around a wearer's body, such as lycra (e.g., Spandex®) and/or neoprene. Elastic, panels and/or stitched seams can be positioned to conform to or follow the contours of various anatomical portions of the wearer's body, such as the wearer's neck and/or chin. The facial covering can comprise padding and/or regions formed from a soft material for comfort and/or to protect the wearer from injury.

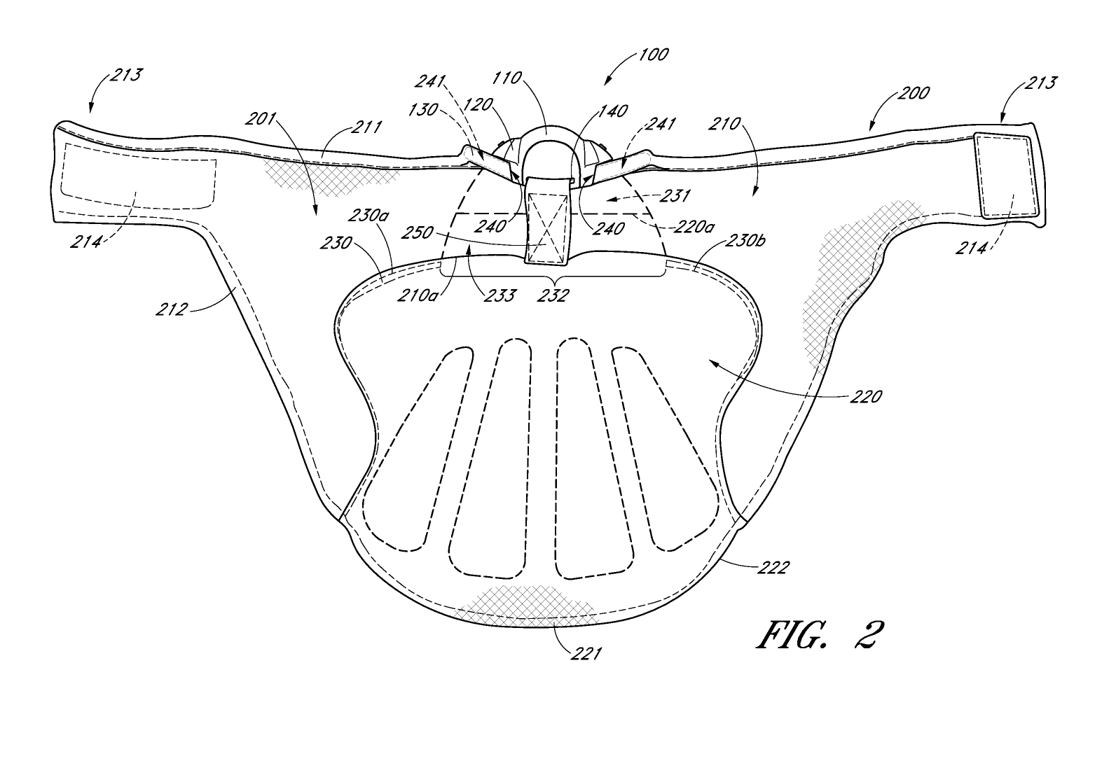

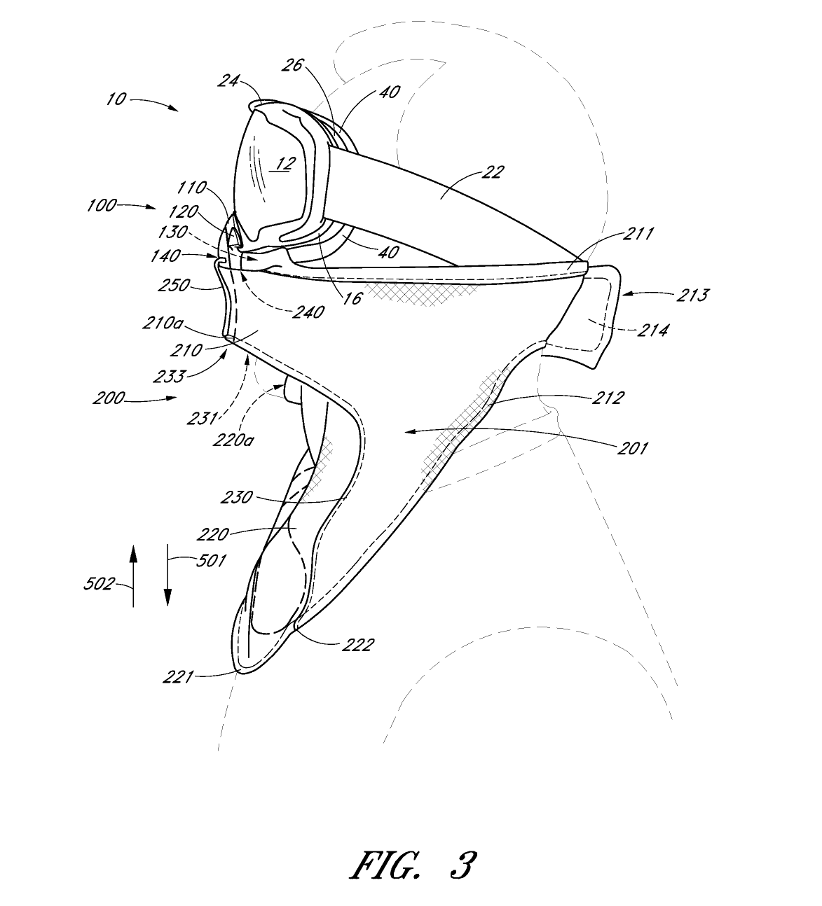

FIG. 2 is a front view of an embodiment of the facemask frame 100 shown in FIG. 1 engaged with an illustrative facial covering (e.g., facemask 200) in a flat position. FIG. 3 is a side view of an embodiment of the facemask frame 100 shown in FIG. 1 engaged with a goggle 10 and facemask 200, in an as-worn position. FIG. 4 is a front view of an embodiment of the facemask frame 100 shown in FIG. 1 engaged with a goggle and a facemask, in an as-worn position.

Referring to FIGS. 1-4, facemask 200 can comprise a body 201 configured to cover a portion of a wearer's face, chin and/or neck. Body 201 can comprise a single, integrally formed piece, or can comprise one or more separate or integrally formed sections attached to each other. For example, body 201 can comprise an upper section 210 attached to a lower section 220. Sections 210, 220 can comprise any of a variety of shapes and sizes, for example, to accommodate an adult or a child. Sections 210, 220 can comprise the same or different materials with respect to each other. For example, lower section 220 can comprise a more permeable material with respect to upper section 210, to allow improved ventilation through lower section 220, or vice versa. Upper section 210 can comprise a material with greater flexibility and/or elasticity than lower section 220, to allow upper section 210 to be stretched and secured around a wearer, or vice versa.

Upper section 210 can be sized and shaped to cover, and in some embodiments, wrap at least partially around, a lower portion of the wearer's face, chin and/or neck. When facemask 200 is engaged with goggle 10, upper sections 210 can cover a portion of goggle 10 (e.g., a lower portion of frame 16 and/or lens 12). Upper section 210 can comprise a top edge 211 and upper side edges 212. Upper side edges 212 can be substantially straight, or, as illustrated in the exemplary embodiment, tapered, to provide a better fit around the neck and face of a user. Top edge 121 can be curved, or substantially straight. Upper section 210 can comprise a pair of laterally-extending members (e.g., straps) 213 with an attachment element 214 configured to hold facemask 200 around a user's neck and/or face. Attachment element 214 can include buckles, snaps, zippers, clasps, buttons, magnets, hook and loop fasteners (e.g., VELCRO®), and any other fastening, adjustment and/or engagement device known or described herein. In some embodiments, members 213 can be connected at their distal ends to form a loop that is pulled over a wearer's head during use.

Lower section 220 can be sized and shaped in any way to cover, and in some embodiments, wrap at least partially around, a portion of a wearer's body, such as a lower portion of a wearer's neck or upper portion of a wearer's torso. Lower section 220 can comprise a bottom edge 221 and lower side edges 222 positioned between bottom edge 221 and upper side edges 212. Edges 221, 222 can be curved, or substantially straight. Lower section 220 can be sufficiently long enough to allow it to be tucked into a wearer's coat, jacket or other garment covering the wearer's torso. Lower section 220 can comprise straps similar to members 213 on upper section 210. Lower section 220 can be formed separately from or integrally with upper section 210. Lower section 220 can be permanently or removably attached to upper section 210, using any of the attachment elements, structures, and/or methods known or described herein. Lower sections 220 and upper section 210 can be separate pieces, or a unitary construction. In embodiments wherein facemask 200 comprises various flexible fabrics, sections 210, 220 can be attached with stitching, adhesive, ultrasonic welding, staples, rivets or the like. In the illustrated embodiment, portions of section 210 and section 220 are stitched to each other along portions of a hem 230. The shape of hem 230 can be varied to provide better fit and comfort to the user. In the illustrated embodiment, hem 230 can comprise a pair of opposed hem portions 230a, 230b that extend medially inwardly with respect to edges 212 and along a lower portion or edge 210a of upper section 210. In the illustrative embodiment, hem 230 comprises other portions that form a curvilinear spline that extends medially from opposed lower points of edges 212, then upwardly (e.g. in a superior direction), and then inwardly towards portions 230a, 230b. In some embodiments, a portion of hem 230 can include stitching that extends only through one of sections 210, 220, to provide an aesthetic hem without attaching that portion of sections 210, 220 to each other.

Hem 230 can extend across some, most, or substantially the entirety of the height and/or width of upper section 210 and/or lower section 220, and can extend continuously or intermittently (e.g., with one or more spaced hem portions). In the illustrated embodiment, hem portions 230a, 230b are spaced with respect to each other, to form a gap 232 therebetween. Space or gap 232 allows a portion of sections 210, 220 to be separable and spaced with respect to each other, to form a channel or opening 231 between sections 210, 220. For example, referring to FIG. 2, gap 232 can allow the lower edge 210a of upper section 210 to be separable with respect to an upper edge 220a of lower section 220. In a preferred embodiment, gap 232 can be a length that is greater than or equal to the length between the distal ends of a corresponding engagement structure, such as arms 130, described further herein. Opening 231 can allow airflow between a posterior portion and anterior portion of sections 210, 220, to provide a breathing passage for a wearer of facemask 200.

Sections 210, 220 can include one or more optional overlapping (or underlying) portions, or flaps, proximate to and/or covering a portion of opening 231 and/or gap 232 to allow a breathing, ventilation, or access passage through a portion of body 201, while still providing protection to the user. For example, the upper edge 220a of lower section 220 can be positioned above (e.g., superior to, in the as-worn position) the lower edge 210a of upper section 210, to form a flap 233 that can cover a portion of channel 231. It will be understood that the edges 220a, 210a are oriented approximately horizontally, and can form an approximately horizontally oriented optional flap 233 (in the as-worn position) for illustrative purposes only. Other embodiments of facemask 200 can include overlapping flaps and/or edges configured in approximately vertical or angled orientations.

Some embodiments of the flaps described herein can be movable between an open and a closed position. In such embodiments, the flap can be configured to allow less flow (e.g., airflow) through a ventilation opening in the closed position with respect to the amount of flow the flap allows through the ventilation opening in the open position. The flap can cover a portion of the ventilation opening in the open and/or the closed position; preferably, the flap covers a greater portion of the ventilation opening in the closed position than in the open position.

For example, FIGS. 2 and 4 illustrate an embodiment of facemask 200 with flap 233 in a closed position. When in a closed position, flap 233 can allow some airflow and access through gap 232 and through channel 231, allowing for some ventilation or access through facemask 200, while providing some protection to the wearer. However, during use, a wearer may desire additional airflow or access through facemask 200. For example, a wearer may want to eat or drink, and/or to allow additional ventilation to defog a goggle if used in combination with facemask 200.

Referring to FIG. 3, lower section 220 can be pulled or moved (e.g., in a downward or inferior direction, as indicated by directional arrow 501), causing the upper edge 220a of lower section 220 and the lower edge 210a of upper section 210 to separate from each other. This movement of edges 210a, 220a with respect to each other causes flap 233 and/or opening 231 to move or stretch with respect to each other, such that flap 233 moves to an open position or assumes an open position relative to the lower section 220. That is, the flap 233 does not necessarily move; however, for convenience, the flap 233 is referred to as having an open position. When flap 233 is in an open position, less of opening 231 is covered by flap 233, allowing additional airflow through opening 231, and/or providing additional access through opening 231. After lower section 220 is released (e.g., moved in an upward or superior direction, as indicated by directional arrow 502), the flap 233 can return to the closed position shown in FIGS. 2 and 4.

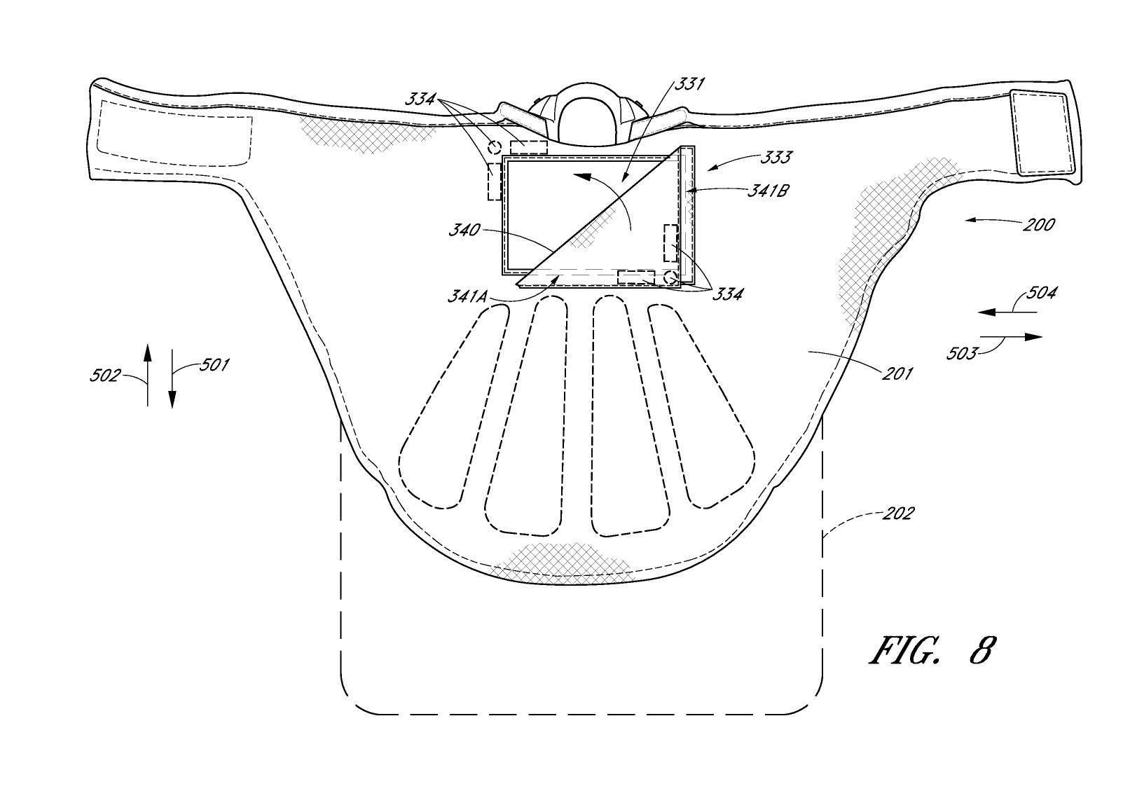

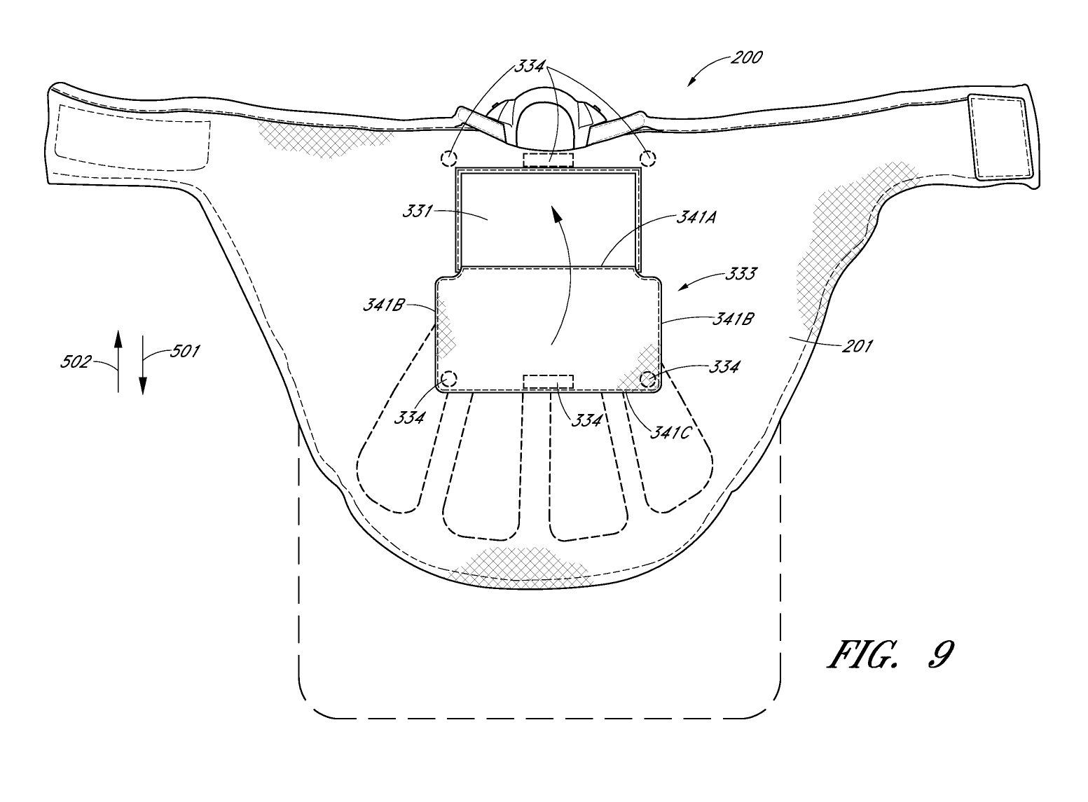

Additional embodiments of flaps that can be implemented with facemask 200 are described further below, with reference to FIGS. 7A-11B.

It will be understood that one or more ventilation openings can extend through body 201 to provide similar function as channel or opening 231, without requiring the structure provided by flap 233, hem 230 and/or hem portions 230a, 230b. For example, one or more openings of any of a number of different shapes and sizes can extend directly through facemask 200 without a flap, for example, through a slit or other opening positioned between or extending through sections 210, 220, to allow ventilation, breathing, or access to a wearer. In some embodiments, a plurality of openings can be implemented to form a mesh, web, or similar structure that provides breathability through facemask 200.

Facemask 200 can engage with goggle 10 and/or facemask frame 100 using any attachment elements, structures or methods known or described herein. For example, a portion of facemask 200 (e.g. edge 211 of upper section 210) can engage with goggle 10 (e.g., a lower portion of frame 16) or a portion of frame 100 (e.g., arms 130, described further herein), using magnets, hook and loop fastening systems, snaps, buttons, etc.

In some embodiments, one or more openings 240 can extend through a portion of upper body 201, such as upper section 210, the openings configured to engage with a corresponding portion of frame 100 or goggle 10. In some embodiments, facemask 200 can comprise a pair of openings 240 extending through a portion of body 201, such as upper section 210. In some embodiments, the openings 240 can be extended (e.g., laterally or medially), to form one or more loops, lumens, hems or channels in an upper portion of upper section 210 to engage with a corresponding portion of frame 100. Channels can be formed through attachment of a preformed loop or channel of material to upper section 210, or can be integrally formed, for example, by folding a portion of upper section 210 and attaching (e.g., removably attaching) a portion of an edge of upper section 210. In some embodiments, a hem can be stitched along a portion of edge 211 to form one or more channels. In a preferred embodiment, a pair of opposed, laterally extending channels 241 with medially facing openings 240 are formed along a portion of body 201 (e.g., upper edge 211), with a space or gap 242 therebetween. Channels 241 can receive and thus engage (preferably, removably engage) with a portion of facemask frame 100 described further herein.

In some embodiments, facemask 200 can attach to frame 100 with one or more optional straps, such as a strap 250. Strap 250 can be permanently or removably attached to upper section 210. Strap 250 can attach to frame 100 using any of the engagement elements, attachment structures and methods described herein; in the illustrated embodiment, strap 250 can form a loop that extends through a slot 140 extending through a portion of frame nosepiece 110. In some embodiments, strap 250 can include an insert, clip, tab, or other structure that engages (e.g., snaps or clips) with slot 140. In the illustrated embodiment, a single strap 250 is approximately centered along the upper edge 211 of upper section 210, to engage with slot 140 on frame 100. However, it will be understood that one or more straps 250 can engage with various portions of frame 100, and/or can be positioned off-center with respect to upper section 210 and/or frame 100. Strap 250 can attach to facemask 200 and/or frame 100 using any of the various attachment mechanisms described herein (e.g., for straps 213). In a preferred embodiment, strap 250 can extend from the upper edge 211 of the facemask 200 to the portion 230a of hem 230, to provide tension on upper section 210 when facemask 200 is being worn. Such tension can facilitate the separation of upper section 210 and lower section 220 at portion 230a, to minimize restriction of airflow through opening 231.

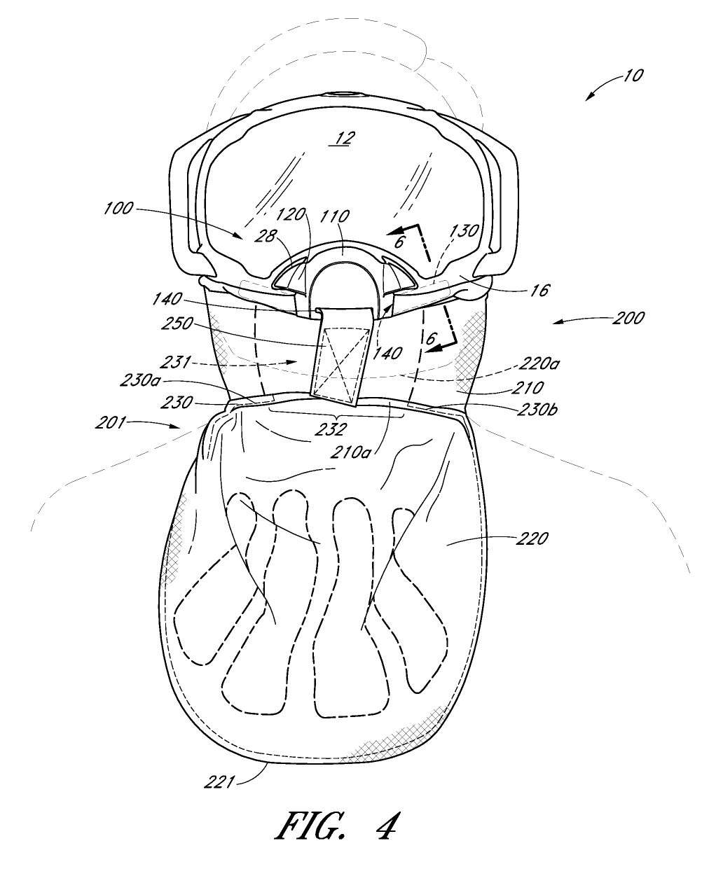

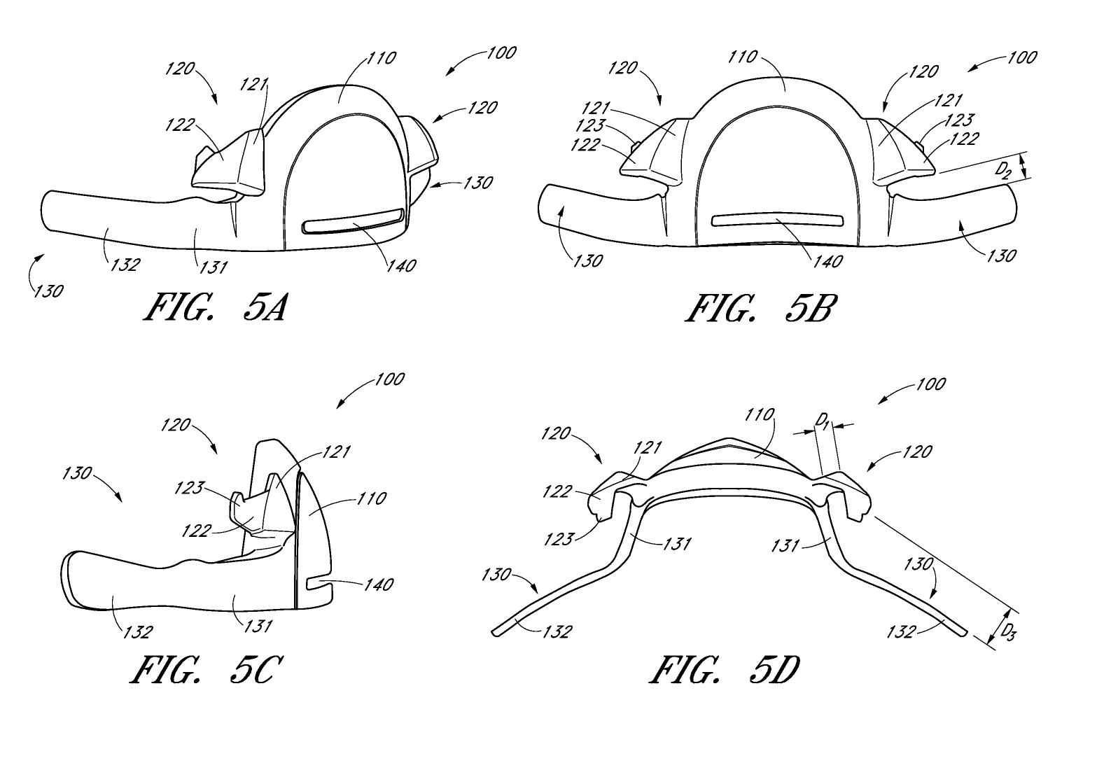

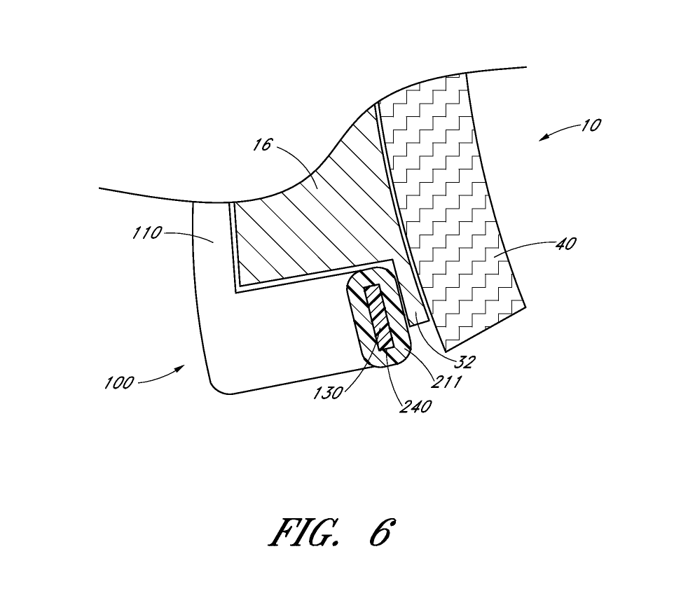

FIGS. 5A-5D are front and right perspective, front, side, and top views, respectively, of an embodiment of the facemask frame 100 shown in FIG. 1. The facemask frame and its components can comprise any of the aforementioned materials described for goggle frame 16, and can comprise the same or different materials with respect to goggle frame 16 and/or each other.

Referring to FIGS. 1-5D, the facemask frame 100 can comprise a frame nosepiece 110 configured to cover and/or removably engage with (e.g., seal) nosepiece 20 on goggle 10, and to cover and protect the wearer's nose. Frame nosepiece 110 can comprise any of a variety of shapes that facilitate such engagement and/or coverage, while providing sufficient room for a wearer's nose (FIGS. 1, 3-4). In some embodiments, nosepiece 110 can be shaped to allow a user to easily grasp frame 100 during attachment and removal of frame 100 to and from goggle 10. Frame nosepiece 110 can comprise a shape that is substantially different from or similar to the approximate shape of medial recess 20. In the illustrated embodiment, nosepiece 110 is an approximately semi-circular or arch-shape that conforms to the approximate shape of medial recess 20. In some embodiments, frame nosepiece 110 can be sized and shaped such that the outer edges of nosepiece 110 overlap (e.g., cover and/or seal) the outer edge of medial recess 20 (e.g., the lateral and/or superior edges of recess 20 on the anterior surface of frame 16). In some embodiments, frame nosepiece 110 can be configured such that its edges overlap (e.g., cover) all or a portion of engagement recesses 28. In such embodiments, frame nosepiece 110 can include one or more of the engagement elements described herein to engage nosepiece 110 with recesses 28.

In some embodiments, frame nosepiece 110 (or portions thereof) can be configured to be inserted into and received by medial recess 20, to provide lateral stability. Medial recess 20 and/or frame nosepiece 110 can comprise one or more protrusions, flanges, shoulders, chamfers, and the like, to facilitate insertion of nosepiece 110 into medial recess 20, and/or engagement therebetween. For example, an annular flange can be configured to extend medially and/or in an inferior direction from the inner surface of medial recess 20, to allow engagement between an anterior surface of the annular flange with a posterior surface of nosepiece 110. In some embodiments, the inner (e.g., medial and/or inferior) surfaces of medial recess 20 and/or the outer (e.g., lateral and/or superior) surfaces of frame nosepiece 110 can comprise any of the engagement elements described herein to removably engage frame nosepiece 110 with medial recess 20.

Referring to FIGS. 1-5D, the frame 100 can include one or more goggle engagement members, shown as arms 120 that can be used to engage the facemask frame 100 with the goggle 10. Arms 120 are not limited to an elongated straight member, and can comprise a variety of elongated, non-elongated, straight, curvilinear, or other shapes and structures that can extend from nosepiece 110 and attach to goggle 10. Arms 120 can extend laterally, medially, and/or in an anterior, posterior, superior and/or inferior direction and/or at various angles with respect to nosepiece 110. Arms 120 and nosepiece 110 can be separately formed and attached to each other using a variety of the attachment methods described herein, or can be a solid or unitary component. Preferably, arms 120 can be positioned proximate to an upper portion of nosepiece 110, to allow sufficient clearance for a facemask engagement member to be attached to nosepiece 110, as described further herein.

Arms 120 can include an engagement portion 122 to engage nosepiece 110 with goggle 10. Engagement portion 122 can include one or more of the engagement elements described elsewhere herein, to facilitate its engagement with goggle 10. In some embodiments, engagement portion 122 can engage with goggle 10 through a press or interference fit (e.g., into recesses 28; FIG. 1), without additional structure. In the illustrated embodiment, engagement portion 122 comprises an engagement element 123 configured as a tab positioned along a portion of engagement portion 122 that can engage with (e.g., using a snap fit) a corresponding slot or groove in goggle 10 (e.g., within recesses 28).

Engagement portion 122 can directly engage with or attach to goggle 10, or can engage thereto with an intermediary structure. An intermediary structure can be attached to (e.g., permanently attached to) goggle 10, and frame 100 can be attached to (e.g., removably) the intermediary structure. Such embodiments can be provided to retrofit an existing goggle with the facemask frame 100. Referring to FIG. 1, optional inserts 300 can be used as intermediary structure to facilitate engagement of facemask frame 100 with a goggle 10. In such an embodiment, inserts 300 can be shaped to be received by, or inserted into, recesses 28, through any of the engagement elements and methods described herein, to attach inserts 300 to frame 16. Insert 300 can also include one or more engagement elements (e.g., such as a groove or slot) to removably attach insert 300 to frame 100 (e.g., to engagement portion 122).

Engagement portion 122 can directly attach to various portions of nosepiece 110. For example, engagement portion can attach directly to nosepiece 110 (e.g., a posterior surface of nosepiece 110), and extend in a posterior direction from nosepiece 110. Such embodiments can allow the edges of nosepiece 110 to overlap with recesses 28, and hide engagement portion 122 from view when frame 100 is engaged with goggle 10. In some embodiments, engagement portion 122 can attach to nosepiece 110 with an intermediary structure, such as an extension portion 121. Extension portion 121 can extend outwardly from nosepiece 110, to separate engagement portion 122 from the edge of nosepiece 110 by a distance D1 (FIG. 5D). Such separation allows portion 122 to engage with goggle frame 16 (e.g., through insertion into recesses 28), and/or allows nosepiece 110 to cover medial recess 22, without interference. Distance D1 can be varied to provide varying levels of stability between nosepiece 110 and goggle frame 16, and/or to correspond to various positions of recesses 28 on goggle frame 16. In some embodiments, D1 can be within a range of between about at least 0.1 inches and/or less than or equal to about 0.75 inches. Further, in some embodiments, D1 can be about 0.25 inches. Extension portion 121 can extend in various directions with respect to nosepiece 110, and can include one or more portions with various directional elements to provide the aforementioned separation between portion 122 and nosepiece 110. In the illustrated embodiment, extension 121 includes one or more portions with directional elements that extend in an anterior, posterior, lateral, superior, and inferior direction.

FIG. 6 is a partial side cross-sectional view of an embodiment of a facemask frame engaged with a goggle and a facemask taken along lines 6-6 of FIG. 4. Referring to FIGS. 1-6, the frame can include one or more facemask engagement members, shown as a pair of arms 130, configured to engage with (e.g., removably) and support facemask 200. Arms 130 are not limited to an elongated straight member, and can comprise a variety of elongated, non-elongated, straight, curvilinear, or other structures that can extend from nosepiece 110 a sufficient length to support facemask 200. Arms 130 and nosepiece 110 can be separately formed and attached to each other using a variety of the attachment methods described herein, or can be a solid or unitary component.

Arms 130 can engage with facemask 200 in a variety of ways, using the various engagement or attachment methods described herein. For example, arms 130 can attach to facemask 200 using adhesive, snaps, hooks, tabs, buttons, and other attachment mechanisms described herein. In some embodiments, arms 130 can be configured to facilitation their insertion into one or more of the aforementioned corresponding loops, lumens, hems or channels (e.g., channels 240; FIGS. 1-4 and 6) formed in facemask 200. It will be understood that arms 130 need not completely extend into channels 240. Moreover, the portion of arms 130 that is received by channels 241 need not be completely surrounded by channels 241 along their engagement therewith. For example, channels 241 can comprise one or more loops into which an arm 130 can be inserted, with intermittent points of non-engagement between arm 130 and channel 240. In a preferred embodiment, arms 130 can be configured such that a length between the points of the proximal attachment of arms 130 to frame nosepiece 110 is less than or equal to the length of gap 232 (FIGS. 1-4).

The arms 130, or portions thereof, can be attached to nosepiece 110 at various positions. Arms 130 can be positioned on a portion of nosepiece 110 (e.g., a lower portion) that allows for sufficient clearance for arms 120. Preferably, arms 130 are attached to nosepiece 110 an inferior distance D2 from the attachment of arms 120 to nosepiece 110, to prevent interference during engagement of frame 100 with goggle 10. Distance D2 can be varied to provide varying levels of stability between nosepiece 110 and frame 100, to correspond to various positions of recesses 28, and/or various size goggle frames. In some embodiments, D2 can be within a range of between about at least 0.05 inches and/or less than or equal to about 0.50 inches. Further, in some embodiments, D2 can be about 0.1 inches.

Arms 130 can extend laterally, medially, and/or in an anterior, posterior, superior and/or inferior direction and/or at various angles along nosepiece 110. Arms 130 can be configured to extend from nosepiece 110 and along a portion of frame 16 (e.g., an inferior edge), when arms 130 are attached to facemask 200 and goggle 10 (FIGS. 3, 4 and 6). Preferably, arms 130 are configured to extend along an anterior portion of frame 16, such as its inferior anterior edge, or an anterior-facing portion of lip 32 (FIG. 6). In some embodiments, arms 130 can sufficiently conform to and contact a portion of frame 16 to seal a portion of facemask 200 against goggle 10 when frame 100 is attached to goggle 10 and facemask 200. In some embodiments, arms 130 can be configured to be biased against a portion of frame 16 when frame 100 is attached to goggle 10 and facemask 200. In some embodiments, arms 130 can be configured such that the portion of arms 130 extending along the goggle frame 16, and the portion of the facemask 200 positioned between arms 130 and goggle frame 16, can contact and/or seal the facemask 200 against the goggle frame 16 without mechanically attaching the facemask 200 and/or the arms 130 to the goggle frame 16 when the arms 120 are engaged with the facemask 100.

Referring again to FIGS. 5A-5D, arms 130 can include an inner portion 131 attached to nosepiece 110, and an outer portion 132 that is attached to and extends from inner portion 131 (e.g., a distal end). Portions 131, 132 can extend laterally, medially, and/or in an anterior, posterior, superior and/or inferior direction and/or at various angles with respect to each other and/or nosepiece 110. Portion 132 is preferably shaped, and configured with respect to portion 131, to extend along an anterior portion of frame 16, and more preferably, an anterior-facing portion of lip 32 (FIG. 6), when facemask frame 100 is attached to goggle frame 16, as described above generally for arm 130. Inner portion 131 can extend outwardly from nosepiece 110, and is configured to separate outer portion 132 from nosepiece 110 and/or from arm 120 by a distance D3. Such separation allows arms 120 and 130 to be spaced anteriorly apart from each other. Such spacing allows arms 130 to seal the facemask 200 along a rear portion of frame 16 (e.g., lip 32), while allowing arms 120 to engage with a front portion of frame 16 (e.g., recesses 28). Distance D3 can be varied to provide varying levels of sealing and/or for various shaped goggles. In some embodiments, D3 can be within a range of between about at least 0.25 inches and/or less than or equal to about 1.25 inches. Further, in some embodiments, D3 can be about 0.5 inches. In some embodiments, D3 can be equal to or greater than the distance between the anterior-facing portion of goggle 10 with which arms 120 engage, and the portion of the goggle frame 16 along which arms 130 extend (e.g., lip 32; FIG. 6). Such embodiments can provide a bias between arms 130 and goggle frame 16, as described elsewhere herein, when facemask frame 100 is engaged with goggle 10 and facemask 200. In some embodiments, D3 can be equal to or greater than the distance between the portion of goggle 10 covered by nosepiece 110, and the portion of the goggle frame 16 along which arms 130 extend. Such embodiments can also provide the aforementioned bias.

Some embodiments of a goggle similar to goggle 10 can be provided that can removably engage directly with a facemask such as facemask 200, without the use of a facemask frame such as frame 100. For example, a pair of arms can extend laterally from a goggle nosepiece, to provide similar structure and function as arms 120 on frame 100, shown, e.g., in FIGS. 5A-5D. Additionally, a goggle can include one or more slots extending through a portion of its frame, similar to slot 140 in nosepiece 110 of frame 100, to which a portion of facemask 200 can attach.

FIGS. 7A-11B show various embodiments of flaps 333 that can be implemented with facemask 200. The embodiments of facemask 200 and the flaps 333 illustrated in FIGS. 7A-11B are similar to those described in FIGS. 1-4, unless otherwise noted. Flaps 333 can comprise a square, rectangular, circular, elliptical, oblong, triangular, or other regular or irregular shape. Flap 333 can comprise any of the materials described herein for body 201, and can comprise the same or different material as body 201. Flap 333 can comprise a permeable material, and/or can include one or more optional openings, apertures or channels extending therethrough, to provide some ventilation when flap 333 is in a closed position.

Flap 333 can be movable between an open and closed position with respect to an opening 331, similar to flap 233 and opening 231 (FIGS. 1-4). It will be understood that any of the various embodiments described herein for the flaps and ventilation openings can be implemented separately or in combination with the structure described to engage facemask 200 with goggle 10 and/or frame 100, such as arms 120 and/or lateral channels 241 (e.g., FIG. 1).

Referring generally to FIGS. 7A-11B, any of a number of different portions (e.g., a corner, edge, perimeter, and/or other portion) of flaps 333 can be attached to body 201 in a variety of orientations. Flap 333 can be configured to move between an open and closed position with respect to opening 331 in a variety of directions, depending on the configuration of the attachment between portions of flap 333 and body 201. For example, flap 333 can be attached to body 201 such that flap 333 can open and close in a horizontal direction (503, 504), and/or a vertical direction (501, 502). In some embodiments, flap 333 can be attached to body 201 such that flap 333 can open and close in an angled or slanted direction that combines such horizontal and vertical movement, depending on the configuration of the attachment between flap 333 and body 201.

Continuing to refer generally to FIGS. 7A-11B, one or more portions (e.g., a corner, edge, perimeter, and/or other portion) of flap 333 can be configured with varying degrees and types of attachment with respect to a portion of body 201. Such embodiments can provide various levels of coverage, breathability, movability and ease of access of flap 333 with respect to ventilation opening 331.

For example, flap 333 can include one or more removable engagement elements, such as a snap, button, hook/loop system, magnet, etc., that can allow a portion of flap 333 to be removably attached to and from body 201, and movable with respect to opening 331. One or more corresponding engagement elements can be positioned on body 201 to attach flap 333 thereto. The engagement elements can be positioned on body 201 such that flap 333 can be removably secured in an open or a closed position, or any intermediary position, with respect to opening 331. In some embodiments, flap 333 can include one or more removable engagement elements configured such that the entirety of flap 333 can be removably attached to and from body 201.

A portion of flap 333 can alternatively or additionally be attached to body 201 with stitching, ultrasonic bonding, or any other semi-permanent or permanent attachment methods known or described herein. Such semi-permanent or permanent attachment structure and methods can provide increased strength along an edge, corner, perimeter, or other portion of flap 333 attached to body 201, to prevent loss of flap 333 during its opening and closing with respect to opening 331.

In some embodiments, flap 333 can include freely movable portions (e.g., a corner, edge, perimeter, and/or other portion) that do not comprise any engagement structure, allowing additional freedom of movement of those portions of flap 333 with respect to body 201. Such freely movable portions can allow a wearer to open and close a portion of flap 333 with respect to opening 331 by simply lifting or moving a portion of flap 333, without having to disengage or engage a removable attachment element, such a snap, button, hook and loop, and the like.

Various embodiments of the facemasks and flaps described herein may combine removable engagement elements, semi-permanent or permanently attached engagement elements, and/or freely movable portions of flap 333, to provide various levels of coverage, breathability, movability and ease of access with respect to ventilation opening 331, and to allow flap 333 to be opened and closed in a variety of manners and directions.

FIGS. 7A and 7B are partial front views of an embodiment of facemask 200 with movable flap 333 shown in a closed and opened position, respectively. Flap 333 is shown as an approximately triangular shape for illustrative purposes only. Opening 331 can be a similar or different shape as flap 333; here it is a similar shape for illustrative purposes only.

In the illustrated embodiment, a pair of engagement elements 334 are attached to a portion of flap 333 and body 201, proximate to a first upper corner 342 of flap 333. Preferably, at least one of elements 334 comprises a magnet, the other comprising a magnet or a ferromagnetic material, configured to removably attach corner 342 to body 201. The engagement elements 334 are positioned to engage corner 342 of flap 333 with body 201 for illustrative purposes only, and can engage other corners, edges, a perimeter, or other portions of flap 333 with body 201.

Flap 333 is attached to body 201 with a semi-permanent or permanent engagement element 339 (e.g., stitching) positioned proximate to a second upper corner 337 of body 201 for illustrative purposes. A third, lower corner 338 of flap 333 is freely-movable with respect to body 201, without any attachment elements.

The illustrated embodiment allows a wearer to move various portions of flap 333 in various directions, to provide varying breathability and access through ventilation opening 331 with varying ease of movement. For example, a wearer can lift freely-movable lower corner 338 in one or more of the directions shown by directional arrows 502-504, moving flap 333 to an open position. Such movement can uncover and thus increase flow through a portion of opening 331, without having to actively disengage a removable engagement element on flap 333 from body 201, such as magnets 334 (or a button, snap, hook/loop system, etc.). When the wearer no longer desires such increased flow, and instead wants additional protection from the elements, the wearer can move corner 338 downwardly or release lower corner 338, and through gravity, external wind, etc., the flap 333 will move downwardly in direction 501 and return to a closed or partially closed position, with decreased flow through opening 331.

A wearer can also disengage and move corner 342 with respect to body 201 and opening 331, in any of directions 501-504, to move flap 333 between an open and closed position. In a preferred embodiment, flap 333 (e.g., engagement element 334 on corner 342) can be detached from a first engagement element 334A (FIG. 7B) on body 201, and attached to a second engagement element 334B on body 201, to removably secure flap 333 in an open position. Engagement element 334B can be positioned along a side, edge, or corner of opening 331 with respect to element 334A, to provide varying levels of flow through opening 331 when flap 333 is held in an open position. Preferably, element 334B is positioned on an opposed side, edge or corner of opening 331 with respect to element 334A, to secure flap 333 in a fully-opened position to provide increased flow through opening 331. In some embodiments, an engagement element can be positioned at an intermediary position (e.g., between opposed corners or edges of opening 331), to hold flap 333 in a partially opened or closed position.

In some embodiments, flap 333 can include a handling portion, or tab 335, configured to allow a wearer to easily grasp and move flap 333. In the illustrated embodiment, tab 335 is positioned on flap 333 proximate to corner 342, but tab 335 can be positioned on or along various corners, edges, etc., of flap 333 to facilitate its movement between an open, closed or partially open and closed position. Tab 335 can include any of a variety of handling structures known or described herein. Preferably, the tab 335 is at least about one and one-half inches in length. In some arrangements, the tab 335 is at least about two inches in length and, in other arrangements, is at least about two and one-half inches in length. Such lengths can facilitate the location and use of the tab 335, even with gloved hands. In some arrangements, the tab 335 extends outwardly of the body 201. In some cases, an entirety or a substantial entirety of the tab 335 can extend outwardly of the body 201. The tab 335 can extend beyond an edge of the corresponding goggle 10 to facilitate location and use of the tab 335.

FIG. 8 is a front view of another embodiment of facemask 200 with a movable flap 333. Flap 333 is an approximately rectangular shape for illustrative purposes only. Flap 333 can be attached to body 201 along one or more portions, such as one or more inferior, superior, and/or lateral edges and/or corners, with any of the aforementioned degrees of attachment. In the illustrated embodiment, flap 333 is semi-permanently or permanently attached (e.g., stitched) along an inferior edge 341A and a lateral edge 341B, such that flap 333 can open and close along a fold 340 to expose or cover opening 331, respectively. The attached portions of edges 341A, 341B can be oriented at various angles, such that fold 340 is slanted, and such that flap 333 opens and closes diagonally with respect to body 201 when facemask 200 is in an as-worn position. Inferior edge 341A can be approximately horizontal with respect to body 201 when facemask 200 is in an as-worn position. Lateral edge 341B can be approximately vertical with respect to body 201 when facemask 200 is in an as-worn position. Facemask 200 can include engagement elements 334 or others known or described elsewhere herein, to removably attach the free edges (edges opposite the inferior edge 341A and lateral edge 341B) to body 201, and secure flap 333 in an open and/or closed position.

The facemask 200 shown in FIG. 8 can also comprise an optional lower neck cover 202 attached to (e.g., removably) and/or extending from body 201 (e.g., in an inferior direction). Cover 202 can provide additional protection to a lower portion of a wearer's neck or an upper portion of a user's chest or torso. Cover 202 can include any of the materials described herein for body 201, and/or can include the same or different material with respect to body 201.

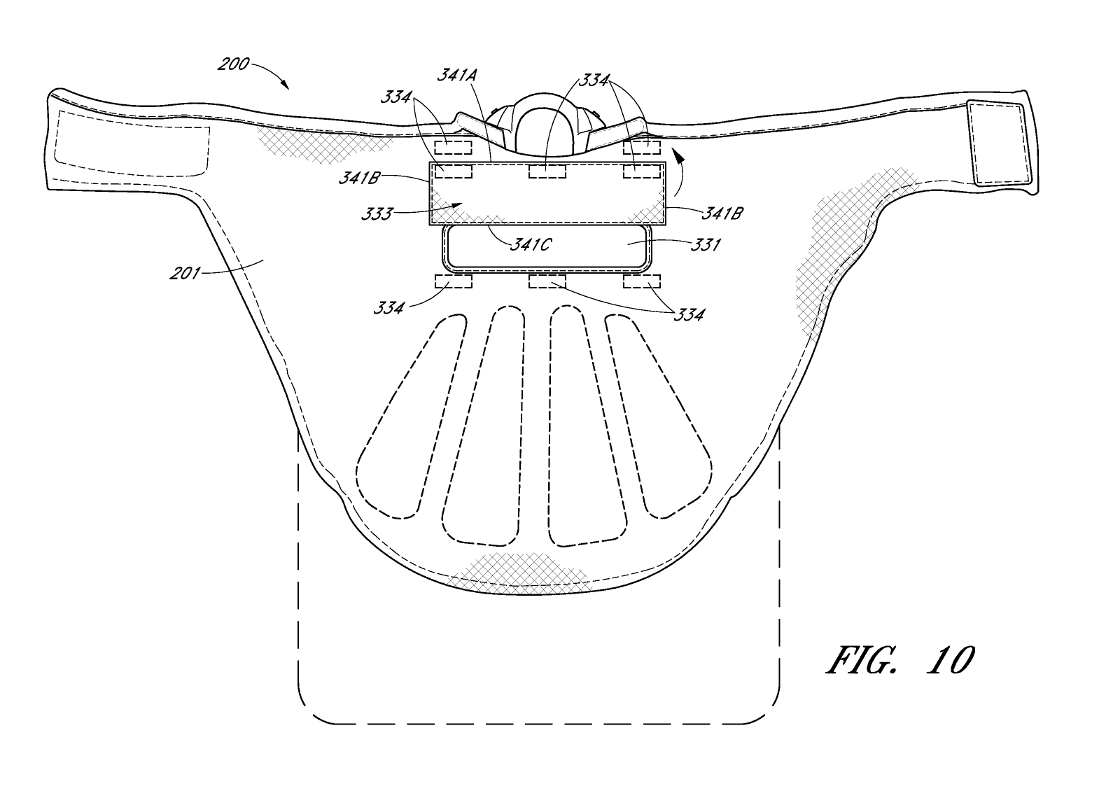

FIG. 9 is a front view of another embodiment of facemask 200 with movable flap 333. The embodiment of flap 333 in FIG. 9 is similar to that shown in FIG. 8, but is shown with inferior edge 341A being attached (e.g. removably, semi-permanently or permanently, e.g., stitched) to body 201, and without lateral edge 341B being stitched to body 201, such that a superior edge 341C of flap 333 can be vertically movable with respect to opening 331. In the illustrated embodiment, lateral edges 341B can be freely movable with respect to opening 331. As such, flap 333 can be moved to an open position by moving superior edge 341C downwardly (direction 501), and can be moved to a closed position by moving superior edge 341C upwardly (direction 502). Such an embodiment can allow flap 333 to be held in an open position, (e.g., by gravity and/or by use of attachment elements 334 attached to a portion of body 201 inferior to opening 331) to prevent flap 333 from inadvertently closing during use.

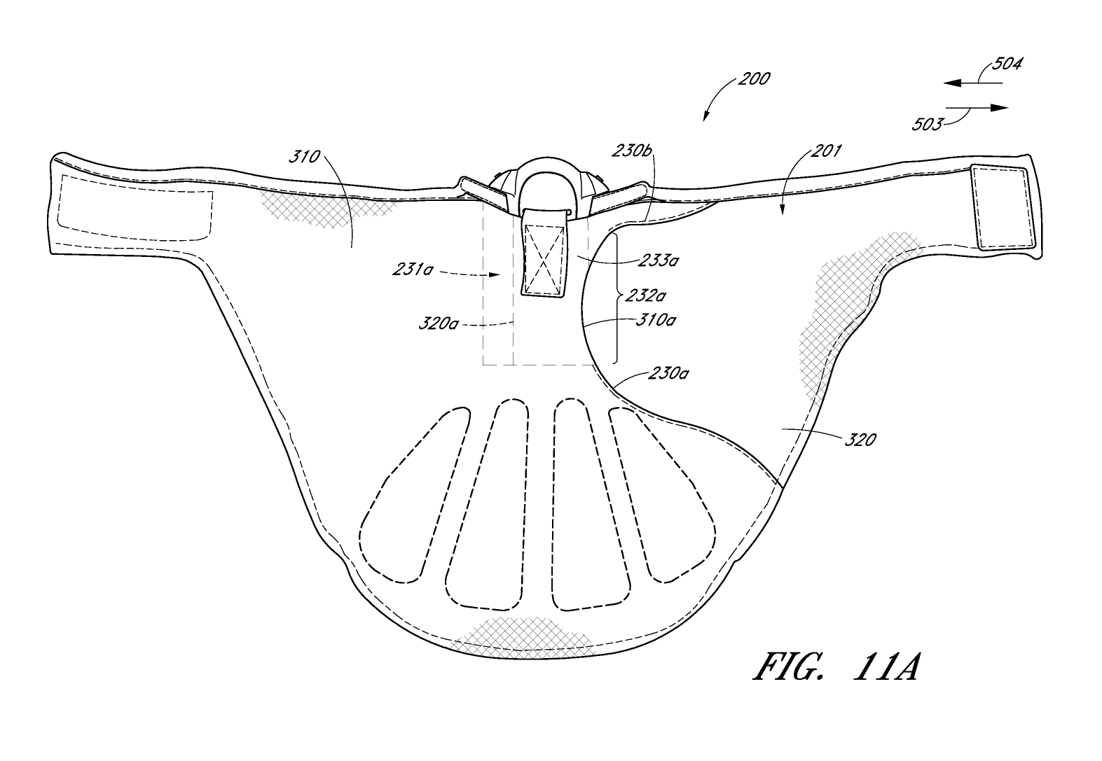

FIG. 10 is a front view of another embodiment of facemask 200 with a movable flap 333. The embodiment of flap 333 in FIG. 10 is similar to that shown in FIGS. 8 and 9, but is shown with superior edge 341C being attached (e.g. removably, semi-permanently or permanently, e.g., stitched) to body 201, and without lateral edge 341B or inferior edge 341A being stitched to body 201, such that inferior edge 341A of flap 333 can be movable with respect to opening 331. In the illustrated embodiment, lateral edges 341B can be freely movable with respect to opening 331. As such, flap 333 can be moved to an open position by moving superior edge 341C upwardly (direction 502), and can be moved to a closed position by moving superior edge 341C downwardly (direction 501). In some embodiments, attachment elements 334 can be positioned along edges 341B, such that flap 333 can be rolled up or down, and attached to body 201 in a rolled position with corresponding attachment elements 334. Such a rolled position can hold flap 333 in a closed, or a partially opened or partially closed position, without inadvertently closing (e.g, through gravity).

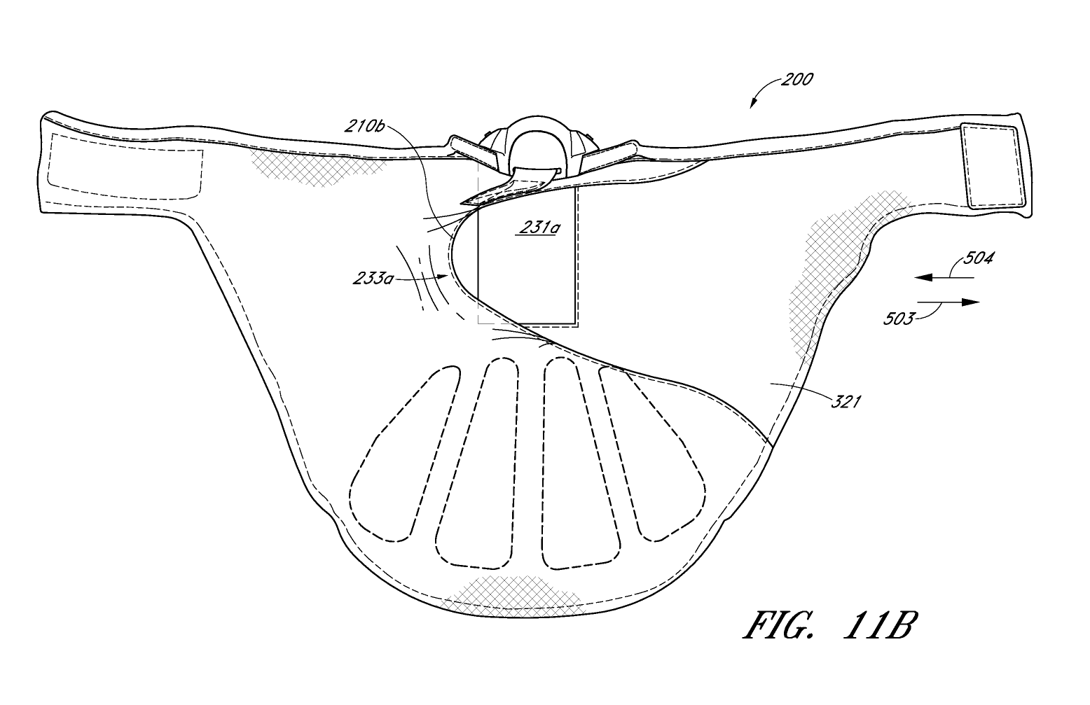

FIGS. 11A and 11B are front views of another embodiment of facemask 200 with a movable flap 233a shown in a closed and opened position, respectively. The facemask 200 shown can be substantially similar to that shown in FIGS. 1-4. In this embodiment, the body 201 of facemask can comprise flap 233a, opening 231a, a first and second lateral sections 310, 320, lateral edges 310a, 320a, and a gap 232a that are similar to flap 233, opening 231, upper and lower sections 210, 220, edges 210a, 220a, and gap 232, respectively, shown in FIGS. 1-4. The main difference is that in the embodiment shown in FIGS. 11A and 11B, these components are oriented approximately vertically when facemask 200 is in an as-worn position, instead of approximately horizontally. Thus, lateral sections 310, 320 can be separated by pulling or moving sections 310, 320 laterally outwardly with respect to each other, in directions 504, 503, respectively. Such movement can cause the lateral edge 310a of first lateral section 310 and the lateral edge 320a of second lateral section 320 to separate from each other (FIG. 11B). This movement of edges 310a, 320a with respect to each other causes flap 233a and/or opening 231a to move or stretch with respect to each other, such that flap 233a moves to an open position. When flap 233a is in an open position, less of opening 231a is covered by flap 233a, allowing additional airflow through opening 231a, and/or providing additional access through opening 231a. After first and/or section lateral sections 310, 320 are released and move in directions 503, 504, respectively, flap 233a can return to the closed position shown in FIG. 11A.

Frame 100, facemask 200, and/or one or more of their components described herein, can include various handling portions to allow a user to easily grasp frame 100, facemask 200 or component(s) during its removal and deployment. These handling portions can include various tabs, knobs, dimples, nipples, surface textures and contours (e.g., concave protrusions and convex recessions), ribs, slots, grooves, and the like, spanning across various portions of frame 100 and/or facemask 200. For example, nosepiece 110, and/or arms 120, 130 can comprise a handling portion to facilitate removal and attachment of facemask frame 100 to and from a goggle. Any of the facemasks 200 can comprise a tab 335, as described above (e.g., FIG. 7A) that can facilitate the movement of flap 333 between an opened and closed position. These handling portions can comprise one or more materials that may improve a user's grip, such as various textured frictional coatings, or resilient materials, such as rubber or foam.

The goggle 10, frame 100, and/or facemask 200 described herein may be attached together at the point of manufacture. Alternatively, the frame and/or facemask can be manufactured as an accessory or replacement part and sold independently without a goggle, helmet shield, etc. Optional inserts 300 can be supplied independently, or with the goggle 10, frame 100, and/or facemask 200, to allow retrofit of the goggle to allow use of frame 100 therewith. Any combination of goggle 10, frame 100, inserts 300, and/or facemask 200 can also be supplied as a kit, wherein the goggle 10, frame 100, inserts 300, and/or facemask 200 can be supplied separately and then assembled by the user.

Although the foregoing embodiments have been described in terms of certain preferred embodiments, other embodiments will become apparent to those of ordinary skill in the art in view of the disclosure herein. Accordingly, the present inventions are not intended to be limited by the recitation of preferred embodiments, but is intended to be defined solely by reference to the appended claims.

What is claimed is:

1. A facemask frame system for attaching a facemask to a goggle, comprising:
a frame nosepiece configured to cover a portion of a goggle nosepiece;
a first pair of opposed arms extending from the frame nosepiece, the first pair of opposed arms configured to removably engage the frame nosepiece with an anterior-facing portion of a goggle frame proximate to the goggle nosepiece;
a second pair of opposed arms extending laterally from the frame nosepiece, the second pair of opposed arms configured to removably engage with a facemask, wherein the second pair of opposed arms are configured to support a portion of an upper edge of the facemask and extend laterally along the goggle frame when the first pair of opposed arms are engaged with the goggle frame and the second pair of opposed arms are engaged with the facemask.

2. The facemask frame system as in claim 1, wherein the second pair of opposed arms are configured to extend laterally along an anterior-facing portion of the goggle frame when the first pair of opposed arms are engaged with the goggle frame and the second pair of opposed arms are engaged with the facemask.

3. The facemask frame system as in claim 1, wherein the second pair of opposed arms are configured to form a seal against the goggle frame with the portion of the facemask when the first pair of opposed arms are engaged with the goggle frame and the second pair of opposed arms are engaged with the facemask.

4. The facemask frame system as in claim 3, wherein the second pair of opposed arms can be configured to extend along and form a seal against the goggle frame without mechanically attaching to the goggle frame when the first pair of opposed arms are engaged with the goggle frame and the second pair of opposed arms are engaged with the facemask.

5. The facemask frame system as in claim 1, wherein the second pair of opposed arms are configured to be biased against the goggle frame when the first pair of opposed arms are engaged with the goggle frame and the second pair of opposed arms are engaged with the facemask.

6. The facemask frame system as in claim 1, wherein the first and the second pair of opposed arms are spaced anteriorly apart with respect to each other by a distance D.

7. The facemask frame system as in claim 6, further comprising the goggle, wherein the distance D is approximately the distance between the anterior-facing portion of the goggle frame proximate to the goggle nosepiece and an anterior-facing surface of a lip extending along a perimeter formed by the goggle frame.

8. The facemask frame system as in claim 7, wherein the lip extends along a posterior perimeter formed by the goggle frame.

9. The facemask frame system as in claim 6, wherein the distance D is approximately within a range of between about at least 0.25 inches and/or less than or equal to about 1.25 inches.

10. The facemask frame system as in claim 1, wherein each of the first pair of opposed arms further comprise a posterior-extending portion with a goggle engagement element configured to removably engage with the anterior-facing portion of the goggle frame.

11. The facemask frame system as in claim 10, wherein the goggle engagement elements are positioned at the approximate distal end of the posterior-extending portions.

12. The facemask frame system as in claim 11, further comprising the goggle, wherein the goggle engagement elements are configured to engage with corresponding facemask frame engagement elements positioned on the anterior-facing portion of the goggle frame.

13. The facemask frame system as in claim 12, wherein the facemask frame engagement elements comprise a pair of recesses formed on opposed sides of the goggle nosepiece.

14. The facemask frame system as in claim 13, further comprising a pair of inserts configured to be received by and engage with the pair of recesses, the inserts further configured to receive the goggle engagement elements, to provide the engagement of the goggle engagement elements with the recesses.

15. The facemask frame system as in claim 1, further comprising the goggle and the facemask.

16. The facemask frame system as in claim 1, wherein each of the second pair of opposed arms include a first portion extending in a posterior direction with respect to the frame nosepiece, and a second portion extending laterally from the first portion.

17. The facemask frame system as in claim 16, wherein the second pair of opposed arms are spaced apart from the frame nosepiece by a distance D corresponding approximately to a distance between an anterior-facing surface of the goggle nosepiece and an anterior-facing surface of a lip extending along the posterior edge of the goggle frame.

18. The facemask frame system as in claim 17, wherein the distance D is approximately 0.5 inches.

19. The facemask frame system as in claim 1, further comprising a slot extending through the frame nosepiece, the slot configured to receive a strap configured to removably engage the facemask with the frame nosepiece.

20. A kit for attaching a facemask to a goggle, comprising:
a pair of inserts configured to be received by and engage with a pair of recesses positioned on an anterior portion of a goggle frame; and
a facemask frame, comprising:
a frame nosepiece configured to cover a portion of a nosepiece on a goggle frame;
a pair of attachment elements extending from the frame nosepiece, the attachment elements configured to removably engage with the inserts; and
a second pair of opposed arms extending laterally from the frame nosepiece, the second pair of opposed arms configured to removably engage with a facemask.

21. A removable facemask system for goggles, comprising:
a goggle, comprising:
a frame with a medial recess configured to cover a portion of a wearer's nose; and
a facemask frame engagement element configured on an anterior portion of the goggle frame proximate to the medial recess;
a facemask with a channel extending along a portion of the facemask; and
a facemask frame for attaching the facemask to the goggle, comprising:
a frame nosepiece configured to cover a portion of the medial recess;
a goggle engagement element extending from the frame nosepiece, the goggle engagement element configured to removably engage with the facemask frame engagement element; and
a pair of opposed arms attached to the nosepiece, the pair of opposed arms comprising a laterally extending portion configured to be inserted into the channel of the facemask, such that the facemask is removably engaged with the facemask frame, wherein the pair of opposed arms are posteriorly disposed on the frame nosepiece with respect to the goggle engagement elements.

22. A facemask system for goggles, comprising:
a facemask, comprising:
a body configured to wrap at least partially around a wearer's face, chin and/or neck;
a pair of medially-facing, laterally-spaced, opposed openings extending through an upper portion of the facemask body, the openings configured to removably engage with a pair of opposed arms extending laterally from a medial portion of a facemask frame or goggle.

23. The facemask system for goggles as in claim 22, further comprising the facemask frame, including the pair of opposed arms, the pair of opposed arms comprising a first pair of opposed arms, the facemask frame further comprising:
a frame nosepiece configured to cover a portion of a goggle nosepiece; and
a second pair of opposed arms extending from the frame nosepiece, the second pair of opposed arms configured to removably engage the frame nosepiece with an anterior-facing portion of a goggle frame proximate to the goggle nosepiece;
wherein the first pair of opposed arms are configured to support a portion of an upper edge of the facemask and extend laterally along the goggle frame when the second pair of opposed arms are engaged with the goggle frame and the first pair of opposed arms are engaged with the facemask through the pair of openings.

24. The facemask system for goggles as in claim 22, wherein the pair of openings extend laterally to form a pair of opposed channels along an upper edge of the facemask body, the channels configured to removably engage the pair of opposed arms.

25. The facemask system for goggles as in claim 22, further comprising one or more ventilation channels extending through a portion of the body.

26. The facemask system for goggles as in claim 25, wherein the body comprises a first body section and a second body section attached to each other along a hem, the hem including a first hem portion spaced from a second hem portion such that at least one of the ventilation channels is formed between the first and second body sections and the first and second hem portions.

27. The facemask system for goggles as in claim 26, wherein a portion of the first body section extends under a portion of the second body section such that the portion of the second body section forms a flap that covers a portion of the at least one ventilation channels.

28. The facemask system for goggles as in claim 25, wherein the one or more ventilation channels comprises a plurality of ventilation openings forming a mesh.

29. The facemask system for goggles as in claim 25, further comprising a flap configured to be movable between an open position and a closed position with respect to at least one ventilation opening, wherein the flap allows less flow through the ventilation opening in the closed position with respect to the amount of flow the flap allows through the ventilation opening in the open position.

30. The facemask system for goggles as in claim 29, further comprising one or more attachment elements configured to attach the flap to a portion of the body in at least one of the open and closed positions.

31. The facemask system for goggles as in claim 30, wherein at least one of the one or more attachment elements comprises a magnet.

32. The facemask system for goggles as in claim 29, wherein the flap comprises a movable edge oriented in an approximately vertical position when the flap is in a closed position and the facemask is in an as-worn position.

33. The facemask system for goggles as in claim 32, wherein the movable edge is oriented in an approximately horizontal position when the flap is in an open position and the facemask is in an as-worn position.

34. The facemask system for goggles as in claim 29, wherein the flap further comprising a tab extending from an edge of the flap and configured to be grasped by a user to move the flap from the closed position to the open position.
